(19) United States
(12) Patent Application Publication
(10) Pub. No.: US 2019/0343835 A1
(43) Pub. Date: Nov. 14, 2019

(54) HAIR LOSS TREATMENT COMPOSITION AND METHOD

(71) Applicants: Leonard Samuel JORDAN, III, Ormond Beach, FL (US); Nava HAJIABADI, Lake Mary, FL (US)

(72) Inventors: Leonard Samuel JORDAN, III, Ormond Beach, FL (US); Nava HAJIABADI, Lake Mary, FL (US)

(21) Appl. No.: 16/184,114

(22) Filed: Nov. 8, 2018

Related U.S. Application Data
(60) Provisional application No. 62/585,637, filed on Nov. 14, 2017.

(51) Int. Cl. A61K 31/506; A61K 31/194; A61K 31/522; A61K 31/07; A61K 31/4415; A61K 33/30; A61K 9/00; A61P 17/14

(52) U.S. Cl. CPC A61K 31/506; A61K 31/194; A61K 31/522; A61P 17/14; A61K 31/4415; A61K 33/30; A61K 9/0014; A61K 31/07

(57) ABSTRACT

In embodiments provided herein, a novel composition for hair growth is provided herein, including a composition of minoxidil, azelaic acid, caffeine anhydrous, retinol, vitamin B6, zinc sulfate, and a pharmaceutically acceptable topical carrier. Further embodiments provided herein include a novel method of making the composition embodiments described herein.

### BACKGROUND

Over 40% of men have noticeable hair loss at 35 years old, by age sixty, the percentage increases to 65%.ⁱ There are 35 million men in the United States who are currently experiencing hair loss.ⁱⁱ There are 21 million women in the United States who are experiencing hair loss.ⁱⁱⁱ Hair loss may occur in the form of male or female pattern baldness, which is characterized by hair receding from the lateral sides of the forehead, and most often occurs in men. Male or female pattern baldness is a hereditary condition, and usually occurs gradually and in predictable patterns.^{iv} Hair loss may also be caused by Alopecia Areata, which is a condition in which the immune system attacks the hair follicles of an individual, and most typically occurs in persons under 20 years old. This condition is marked by loss of hair in clumps, leaving bald patches on the scalp or other portion of the body. Hair loss is not restricted to a particular gender or a particular age group.

Hair loss is often treated in one of two ways, either by stimulating the hair follicle to grow the hair, or by inhibiting the production or buildup of DHT in the hair follicle, which may cause hair loss. Currently available products for treating hair loss in both men and women leave the hair greasy, produce buildup in the hair over time, causes scalp irritation and burning, and/or are marked by a distinct foul smell.

### SUMMARY

In embodiments provided herein, a novel composition for hair growth is provided herein, including a composition of minoxidil, azelaic acid, caffeine anhydrous, retinol, vitamin B6, zinc sulfate, and a pharmaceutically acceptable topical carrier for the treatment of patients suffering from hair loss. Further embodiments provided herein include a novel method of making the composition embodiments described herein, and methods for reducing hair loss and/or enhancing or initiating hair growth in a subject.

### DETAILED DESCRIPTION

For the purposes of promoting an understanding of the principles and operation of the invention, reference will now be made to the embodiments described in the claims and specific language will be used to describe the same. It will nevertheless be understood that no limitation of the scope of the invention is thereby intended, such alterations and further modifications in the illustrated composition, and such further applications of the principles of the invention as illustrated therein being contemplated as would normally occur to those skilled in the art to which the invention pertains.

It is to be noted that the terms “first,” “second,” and the like as used herein do not denote any order, quantity, or importance, but rather are used to distinguish one element from another. The terms “a” and “an” do not denote a limitation of quantity, but rather denote the presence of at least one of the referenced item. The modifier “about” used in connection with a quantity is inclusive of the stated value and has the meaning dictated by the context (e.g., includes the degree of error associated with measurement of the particular quantity). It is to be noted that all ranges disclosed within this specification are inclusive and are independently combinable.

Furthermore, to the extent that the terms “including,” “includes,” “having,” “has,” “with,” or variants thereof are used in either the detailed description and/or the claims, such terms are intended to be inclusive in a manner similar to the term “comprising.” Moreover, unless specifically stated, any use of the terms first, second, etc., does not denote any order, quantity or importance, but rather the terms first, second, etc., are used to distinguish one element from another.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. As a non-limiting example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 7.

All percentages, parts and ratios are based upon the total weight of the composition of the present invention, unless otherwise specified. All such weights as they pertain to the listed ingredients are based on the active level and, therefore, do not include carriers or by-products that may be included in commercially available materials, unless otherwise specified.

The terms “grow” or “growth” as used herein with respect to hair means the growth or regrowth of hair. Accordingly, the terms “growth” and “regrowth” are used interchangeably with respect to growing hair or respect to actives for growing hair.

The term “effective amount” as used herein means an amount of a compound or composition such as a topical or system active sufficient to significantly induce a positive benefit, for example, hair growth, but low enough to avoid serious side effects, i.e., to provide a reasonable benefit to risk ratio, within the scope of sound judgment of the skilled artisan.

As used herein, the terms “subject”, “user” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human.

### Pharmaceutically Acceptable Topical Carriers

The topical compositions described herein include formulations suitable for topical application to skin and scalp. The term “topical” as used herein includes the use of a composition and, in some non-limiting embodiments, a composition with a suitable pharmaceutical carrier. In some non-limiting embodiments, the combination of the composition and the suitable pharmaceutical carrier is applied according to a method described in embodiments herein at the site of hair loss or reduced hair growth to promote hair proliferation.

The term “pharmaceutically acceptable” as used herein, denotes compatibility with the active ingredients contemplated herein. The pharmaceutically acceptable topical carriers are also compatible with the skin, scalp and any keratinous substrates.

Various ingredients are combined in the composition embodiments described herein to achieve the enhanced hair growth by stimulation of the hair follicle to produce hair and decreased DHT production in the hair follicle or on the skin of the subject. Such ingredients that may be included in the pharmaceutically acceptable carrier described in embodiments herein may include humectants used to preserve moisture, surfactants to lower the surface tension between two liquids, or between a liquid and a solid. Surfactants may serve as detergents, wetting agents, emulsifiers, foaming agents and/or dispersants. Moisturizers or emollients may be provided as complex mixtures of chemical agents designed to make the external epidermal layers softer and more pliable. These agents may increase hydration of the skin by reducing evaporation. The solvents described herein are used as dissolving agents. Acidifying agents are used to lower a pH of a spray solution, and are typically derived from phosphoric acid or blends of phosphate ester anionic surfactants containing free phosphoric acid. In a non-limiting embodiment, the composition embodiments described herein may be delivered to a user's scalp in a liquid form via a dropper.

In one embodiment, the pharmaceutically acceptable carrier may include a combination of two or more of: a nonionic surfactant, such as decyl glucoside, in one non limiting embodiment, a solvent, such as denatured ethanol in one non-limiting embodiment, a chelating agent, such as disodium EDTA, an energy source for hair growth and cellular activity, such as glucose, a component to increase the ability of the hair to bind moisture and improve hair strength, also reducing damage due to bleach, perming or combing, such as hydrolyzed soy protein, an acidifying agent such as lactic acid, a humectant/emollient, such as panthenol, an emulsifying agent such as PEG-8 caprylic/capric glycerides, an antimicrobial preservative, such as phenoxyethanol, a surfactant or preservative such as polyquaternium 10, 78, 11, an emollient and penetration enhancer such as propranediol, an emollient such as propylene glycol, an antimicrobial preservative such as sodium benzoate, a humectant such as sodium lactate, an antioxidant, including for example, sodium metabisulfite, a humectant such as glycerin, a solvent, such as for example, water, and another humectant such as zinc Pca, as well as pisum sativum (pea) sprout extract.

In another non-limiting embodiment, the pharmaceutically acceptable carrier may include at least one of: butylated hydroxytoluene (BHT), propylene glycol, caprylic/capric triglyceride, alcohol (95%),benzyl alcohol, ethoxy diglycol, dimethyl sulfoxide, acetic acid, and water.

In some examples, the pharmaceutically acceptable carrier may include between 0.05-0.2 g butylated hydroxytoluene (BHT), may optionally include 0.01-6 ml propylene glycol, may optionally include 0.01-6.0 ml caprylic/capric triglyceride, may include 6.0-33 ml alcohol (95%), 18-33 ml benzyl alcohol, 6.0-15.0 ml ethoxy diglycol, dimethyl sulfoxide, acetic acid, and optionally water in an amount sufficient to fill to quantity sufficient. Additionally, the pharmaceutically acceptable carrier may optionally include poly 80, in an amount ranging 0.01-6.0 ml.

The pharmaceutically acceptable carrier may vary in terms of its composition as described above. Some of the ingredients are optional, as aforementioned, and the amount of each ingredient may vary depending on the result to be achieved. For example, for a sensitive scalp, lower concentrations of alcohol and benzyl alcohol are used to decrease irritation, in some of these examples, a higher concentration of water and/or propylene glycol may be included. For quicker drying composition, a larger concentration of alcohol and lower concentration of propylene glycol, BHT, and poly 80 will be included within the ranges provided above. Therefore, within the provided ranges, the ingredients of the pharmaceutically acceptable topical carrier can be adjusted based on particular needs of the individual. In some embodiments, therefore, the individual components of the composition may be customizable based on the particular needs of the patient, allowing mass customization of the composition. The inventor has discovered, for the first time herein, novel composition and method embodiments for treating hair loss and/or stimulating hair growth in an individual. The inventor has identified an effective composition, including a novel combination of ingredients, combined in such a manner so as to prevent the residue buildup typically found in hair loss treatment products, decrease or eliminate scalp irritation and eliminate the greasy-hair texture consistently provided by other hair loss treatment products due to the ingredients used therein, while effectively stimulating hair growth, minimizing DHT production which is often the main cause of hair loss, and preventing the odorous scent typically found in hair growth products on the market.

The inventor has discovered herein, a novel method and formulation for treating and/or preventing hair loss in a subject by stimulating the hair follicle to grow hair in combination with inhibiting the production of DHT in the hair follicle, in one embodiment. The inventor has further discovered a formulation for hair lost treatment that can be used without the side effects known to be associated with other hair loss treatments.

The inventor has identified that a combination of caffeine and minoxidil provides the most effective hair growth stimulator, and has further identified that the addition of retinol allows the combination of ingredients in the composition to be absorbed more readily by the scalp of a subject. An effective amount of azelaic acid, zinc sulfate and vitamin B6, in combination, is particularly beneficial in inhibiting DHT production. The resulting combination of ingredients in the composition for hair growth provides a pleasant, non-odorous, non-greasy consistency that is both easily washable with a shampoo preventing residue build up and effectively treating hair loss. Zinc sulphate and azelaic acid is effective on 5 alpha-reductase, an enzyme which catalyzes the conversion of testosterone to 5a-DHT.^{v} The combination of zinc sulphate and azelaic acid inhibits 5 alpha-reductase activity. The combination results in an additive effect on inhibition of the enzymatic activity of 5 alpha-reductase. Addition of vitamin B6 to the zinc sulphate and azelaic acid combination potentiates the inhibitory effect of the zinc sulphate.^{vi} The resulting combination of zinc sulphate, azelaic acid and vitamin B6 results in a potent 5 alpha-reductase inhibitor, which is effective in low concentrations.^{vii} This combination is effective in inhibiting 5 alpha-reductase activity by 90%. Zinc sulphate, azelaic acid and vitamin B6 has been identified as being effective in blocking DHT production, and caffeine and minoxidil effectively stimulate hair growth.

Caffeine is a potent stimulator for hair growth. Caffeine can be used to counteract DHT-induced miniaturization of the hair follicle and inhibit phosphodiesterase, to increase cAMP levels in cells, promoting cell proliferation via metabolism stimulation.^{viii} In addition to its effects in inhibiting phosphodiesterase, caffeine is also an effective component or adjuvant in the treatment of hair loss by improving barrier function by substantially reducing transepidermal water loss in individuals, enhancing follicular penetration and stimulating and promoting hair growth.^{ix-x} Minoxidil is a widely approved hair loss treatment, applied topically to stop hair loss in individuals with androgenetic alopecia.^{xi-xii} Retinol, Retin-A and Vitamin A is known for opening pores of the skin and can be used in conjunction with other ingredients to enhance skin penetration. Retinol assists in increasing the absorptive ability of the skin and/or hair follicle, to enhance the receipt of the combination by the subject. Retinol is also known to regulate hair follicle growth through biological manipulation of the immune system. Topical retinol has been shown to increase blood flow to hair follicles and initiate new blood vessel formation.^{xiii}

The consistency of the combination is essential in achieving reduction to hair loss, and enhanced hair growth in a subject. The absorption of the combination depends, in part, on its consistency. Oil-based formulas leave the scalp greasy, and are less effective in treating hair loss, due to its inhibited mobility through the hair shaft. A lighter, more fluid consistency allows the product to move through the hair shaft and be absorbed more readily by the scalp and into the hair follicle. Therefore, a water-based consistency provides an enhanced treatment for hair loss. The consistency of the non-oily base of the combination, in addition to the retinol provides an optimal combination for enhancing the beneficial effects of the combination on the subject.

Furthermore, the inventor has discovered that the particular combination of the composition described herein achieves optimal effects in decreasing or eliminating DHT production, increasing hair growth, and enhancing penetration of the composition, while avoiding the greasy effects, product build up or residue left on the scalp, or the burning of the scalp typically found with hair loss products containing any one of these ingredients.

In a non-limiting embodiment, a composition for hair growth is provided herein. The composition may include a combination of minoxidil, azelaic acid, caffeine, at least one of Retinol, Retin-A, or Vitamin A, vitamin B6, zinc sulfate, and a pharmaceutically acceptable topical carrier.

In one non-limiting embodiment, the composition may include 1-5 grams (g) of minoxidil, 0.1-2 g of azelaic acid, 0.5-2.5 g of caffeine (i.e., caffeine anhydrous), 0.001-0.030 grams of retinol, 0.1-2.5 g Vitamin B6, and 0.1-2 g zinc sulfate. In one non-limiting embodiment, the composition may include 5% minoxidil, in other non-limiting embodiments, the composition may include 2% minoxidil. In some non-limiting examples, 5% minoxidil is used in the composition administered to male subjects, and 2% minoxidil is used in the composition administered to female subjects. In one non-limiting example, 2% caffeine may be preferred.

In another non-limiting embodiment, a method of making a composition for hair growth is provided. The method includes combining minoxidil, azelaic acid, caffeine, at least one of Retinol, Retin-A, or Vitamin A, vitamin B6, and zinc sulfate. In one non-limiting embodiment, the ingredients may be combined in powder form. The method further includes triturating the combination into a mixture, and adding the mixture to a pharmaceutically acceptable topical carrier to substantially dissolve the mixture therein. The method may include the additional step of increasing the amount of the mixture and the carrier to a predetermined volume. This may occur, in one non-limiting embodiment, by increasing the amount of the carrier, or various components of the carrier, for example, increasing the water volume in the mixture. In one example, the predetermined volume may include between 50-70 ml. In a more particular example, the predetermined volume may include 60 ml, or 2 oz. In a further embodiment, the method may include dissolving the mixture within the pharmaceutically acceptable topical carrier with a sieve. In a further non-limiting embodiment, the sieve may include a mesh sieve. In still a further embodiment, the combination is triturated into a homogenous mixture during the triturating step.

In another embodiment, a method for treating hair loss is provided. The method includes applying to an affected area of hair and/or skin the composition described herein, including minoxidil, azelaic acid, caffeine, at least one of Retinol, Retin-A, or Vitamin A, vitamin B6, and zinc sulfate, and a pharmaceutically acceptable carrier, wherein the composition is effective to decrease hair loss and/or enhance hair growth in the affected area. In yet another embodiment, a method for initiating hair growth is provided, the method including applying a composition including minoxidil, azelaic acid, caffeine, at least one of Retinol, Retin-A, or Vitamin A, vitamin B6, and zinc sulfate, and a pharmaceutically acceptable carrier to an affected area of skin, wherein the composition is effective to initiate hair growth in the affected area.

In one non-limiting example, a composition in accordance with the invention for administration to a subject may be prepared as follows:

$$\begin{matrix}\text{caffeine (anhydrous)} & \text{0.5-2.5 grams} \\ \text{minoxidil} & \text{0.5-5 grams} \\ \text{pyridoxine hydrochloride (vitamin} \text{B} \text{6)} & \text{0.1-2.5 grams} \\ \text{vitamin} \text{A} \text{acetate (0.05\%) or retinol} & \text{0.001-0.030 grams} \\ \text{Zinc sulfate} & \text{0.1-2.0 grams} \\ \text{Azelaic acid} & \text{0.1-2 grams}\end{matrix}$$

The composition may further include two or more of the following, in one non-limiting embodiment:

$$\begin{matrix}\text{butylated hydroxytoluene (BHT)} & \text{0.06-0.1 g} \\ \text{propylene glycol} & \text{0-6.0 ml} \\ \text{Caprylic/capric triglyceride} & \text{0-6.0 ml} \\ \text{Alcohol (95\%)} & \text{6.0-33.0 ml} \\ \text{Benzyl alcohol} & \text{18-33.0 ml} \\ \text{Ethoxy diglycol} & \text{6.0-15.0 ml} \\ \text{poly 80} & \text{0-6.0 ml} \\ \text{water} & \text{0 ml -q.s. (quantity sufficient) to 60 mL}\end{matrix}$$

In some non-limiting embodiments, the composition comprises a water base which prevents residue build up at the root of the hair, and provides effective delivery and absorption of the composition embodiments described herein.

In other embodiments, the composition may include the following components:

1-3% caffeine (anhydrous) in one specific non-limiting embodiment 2%;

1-7% minoxidil, in one specific embodiment, 2% minoxidil for women or 5% minoxidil for men;

0.5-2% pyridoxine hydrochloride (vitamin B6), in one non-limiting embodiment, 1%;

0.001-0.05% vitamin A acetate (0.05%) or retinol, in one non-limiting embodiment, 0.025%;

0.5-2% Zinc sulfate, in one non-limiting embodiment, 1%

0.5-3% Azelaic Acid, in one non-limiting embodiment, 1%.

In certain embodiments, the compositions of the present invention may, optionally, further include active agent selected from the group consisting of additional hair growth actives, anti-acne agents, antimicrobial agents, anti-fungal agents, antibiotic or antiseptic agents, antipsoriatic agents, anti-viral agents, anti-seborrea agents, anti-dandruff agents, active agents for treating keratosis pilaris, anti-inflammatory agent, vasodilators, UV absorbers and anti-cancer agents.

In an embodiment, a method of making a composition for hair growth is provided. The method includes combining in powder form: minoxidil; azelaic acid; caffeine; at least one of Retinol, Retin-A or Vitamin A; vitamin B6; zinc sulfate; and triturate combination into a mixture. The method further includes adding the mixture to a pharmaceutically acceptable topical carrier to dissolve the mixture therein; and filling to volume. Volume may be vary; however, in some non-limiting embodiments, volume includes 60 ml. In other examples, volume may include 100 ml.

The method may further include dissolving the mixture within the pharmaceutically acceptable topical carrier with a sieve, in one embodiment. In a further non-limiting step, the method may include during the triturating step, triturating the combination into a homogenous mixture.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, any materials, methods, and examples described herein are illustrative only and are not intended to be limiting.

It should be borne in mind that all patents, patent applications, patent publications, technical publications, scientific publications, and other references referenced herein are hereby incorporated by reference in this application in order to more fully describe the state of the art to which the present invention pertains.

Reference to particular chemicals, ingredients, buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

### REFERENCES

ⁱRelevant Research, Inc. International Society of Hair Restoration Surgery. Statistic Brain. Research Conducted Aug. 30,2016 (http://www.statisticbrain.com/hair-loss-statistics/)
ⁱⁱId.
ⁱⁱⁱId.
^{iv}Mayo Clinic at: http://www.mayoclinic.org/diseases-conditions/hair-loss/basics/causes/CON-20027666
^{v}Stamatiadis D, et al., Inhibition of 5 alpha-reductase activity in human skin by zinc and azelaic acid. Br J Dermatol. 1988 November; 119(5):627-32.
^{vi}Stamatiadis D, et al., Inhibition of 5 alpha-reductase activity in human skin by zinc and azelaic acid. Br J DErmatol. 1988 November; 119(5):627-32.
^{vii}Id.
^{viii}Fischer T W, Hipler U C, Elsner P. Effect of caffeine and testosterone on the proliferation of human hair follicles in vitro. Int J Dermatol. 2007; 46:27-35.
^{ix}Brandner J M, Behne M J, Huesing B, Moll I. Caffeine improves barrier function in male skin. Int J Cosmet Sci. 2006; 28:343-7.
^{x}Otberg N, Patzelt A, Rasulev U, Hagemeister T, Linscheid M, Sinkgraven R, et al. The role of hair follicles in the percutaneous absorption of caffeine. Br J Clin Pharmacol. 2007; 65:488-92.
^{xi}Olsen, e. et al., A randomized clinical trial of 5% topical minoxidil versus 2% topical minoxidil and placebo in the treatment of androgenetic alopecia in men. JAAD September 2002: 47:3:377-385.
^{xii}Lucky, A. W. A randomized, placebo-controlled trial of 5% and 2% topical minoxidil solutions in the treatment of female pattern hair loss. J Am Acad Dermatol. 2004 April; 50(4): 541-53.
^{xiii}Rogers, N. E., & Avram, M. R. (2008). Medical treatments for male and female pattern hair loss. Journal of the American Academy of Dermatology, 59(4), 547-566. doi:10.1016/j.jaad.2008.07.001
Read more at https://www.nicehair.org/topical-retinol-for-hair-growth/#0uxvs3jj4Agdg1OQ.99

What is claimed is:

1. A composition for hair growth, comprising:
minoxidil;
azelaic acid;
caffeine;
at least one of Retinol, Retin-A, or Vitamin A;
vitamin B6;
zinc sulfate; and
a pharmaceutically acceptable topical carrier.

2. The composition of claim 1, wherein the pharmaceutically acceptable topical carrier comprises two or more of the following: Decyl glucoside, Denaturated ethanol, Disodium EDTA, Glucose, Glycerin, Hydrolyzed soy protein, Lactic acid, Panthenol, PEG-8 Caprylic/Capric Glycerides, Phenoxyethanol, Pisum Sativum (Pea) Sprout Extract, Polyquaternium 10, 78, 11, Propranediol, Propylene Glycol, Sodium Benzoate, Sodium Lactate, Sodium Metabisulfite, glycerin Water, and Zinc Pca.

3. The composition of claim 1, comprising 0.5-5 grams minoxidil.

4. The composition of claim 1, comprising 0.1-2 grams azelaic acid.

5. The composition of claim 1, comprising 0.5-2.5 grams of caffeine anhydrous.

6. The composition of claim 1, comprising 0.001-0.030 grams of retinol.

7. The composition of claim 1, comprising 0.1-2.5 grams of Vitamin B6 (pyroxidine hydrochloride).

8. The composition of claim 1, comprising 0.1-2.0 grams of zinc sulfate.

9. A method of making a composition for hair growth, comprising:
combining in powder form:
minoxidil;
azelaic acid;
caffeine;
at least one of Retinol, Retin-A or Vitamin A;
vitamin B6;
zinc sulfate; and
triturate combination into a mixture;
add the mixture to a pharmaceutically acceptable topical carrier to dissolve the mixture therein; and
fill to volume.

10. The method of claim 9, further comprising dissolving the mixture within the pharmaceutically acceptable topical carrier with a sieve.

11. The method of claim 9, wherein during the triturating step, the combination is triturated into a homogenous mixture.

12. The composition of claim 1, wherein the pharmaceutically acceptable topical carrier comprises two or more of the following:
butylated hydroxytoluene (BHT);
propylene glycol;
caprylic/capric triglyceride;
alcohol (95%);
benzyl alcohol;
ethoxy diglycol;
dimethyl sulfoxide;
poly 80
acetic acid; or
water.

13. A method for treating hair loss, comprising:
applying to an affected area of hair and/or skin, the composition of claim 1, wherein the composition is effective to decrease hair loss and/or enhance hair growth in the affected area.

14. A method for initiating hair growth, the method comprising:
applying to an affected area of skin, the composition of claim 1, wherein the composition is effective to initiate hair growth in the affected area.

15. The composition of claim 1, comprising 1-3% caffeine.

16. The composition of claim 1, comprising 1-7% minoxidil.

17. The composition of claim 1, comprising 0.5-2% pyridoxine hydrochloride.

18. The composition of claim 1, comprising 0.001-0.05% vitamin A acetate (0.05%) or retinol.

19. The composition of claim 1, comprising 0.5-2% Zinc sulfate.

20. The composition of claim 1, comprising 0.5-3% Azelaic Acid.
